(19) United States
(12) Patent Application Publication
(10) Pub. No.: US 2020/0085296 A1
(43) Pub. Date: Mar. 19, 2020

(54) Eye state detection system and method of operating the same for utilizing a deep learning model to detect an eye state

(71) Applicant: ArcSoft Corporation Limited, Hangzhou City (CN)

(72) Inventors: PU ZHANG, Hangzhou City (CN); WEI ZHOU, Hangzhou City (CN); Chung-Yang Lin, Hangzhou City (CN)

(21) Appl. No.: 16/217,051

(22) Filed: Dec. 12, 2018

(30) Foreign Application Priority Data
Sep. 14, 2018 (CN) 201811071988.5

(51) Int. Cl. A61B 3/113; G06F 3/01; G02B 27/01

(52) U.S. Cl. CPC A61B 3/113; G06F 3/0304; G02B 27/017; G06F 3/013

(57) ABSTRACT

An eye state detection system includes an image processor and a deep learning processor. After the image processor receives an image to be detected, the image processor identifies an eye region from the image to be detected according to a plurality of facial feature points, the image processor performs image registration on the eye region to generate a normalized eye image to be detected, the deep learning processor extracts a plurality of eye features from the normalized eye image to be detected according to a deep learning model, and the deep learning processor outputs an eye state in the eye region according to the plurality of eye features and a plurality of training samples in the deep learning model.

### BACKGROUND OF THE INVENTION

### 1. Field of the Invention

The invention relates to an eye state detection system, and in particular, to an eye state detection system utilizing a deep learning model to detect an eye state.

### 2. Description of the Prior Art

Owing to growing functionalities of mobile phones, mobile phone users frequently use the mobile phones for capturing images, recording everyday life, and image sharing. In order to facilitate users to capture satisfactory images, in the conventional art, mobile devices are equipped with functions such as eye closure detection for photographing to prevent the users from capturing an image of a person with an eye closed. Further, the eye closure detection technology can be applied in a driving auxiliary system. For example, the eye closure detection technology can be used to determine a driver fatigue situation by detecting eye closure of a driver.

In general, in an eye closure detection process, eye feature points are first extracted from an image, and then information of the eye feature points are compared against a default value to determine whether a person in the image has closed his eyes. Since everybody's eyes are different in shape and size, the eye feature points detected during eye closure may have considerable differences. Furthermore, eye closure detection may fail owing to a part of an eye being hidden by a particular posture of a person, ambient light interference, or eyeglasses worn by a person, leading to unfavorable robustness of eye closure detection, and failing to meet requirements of users.

### SUMMARY OF THE INVENTION

In one embodiment of the invention, a method of operating an eye state detection system is provided. The eye state detection system comprises an image processor and a deep learning processor.

The method of the operating the eye state detection system comprises the image processor receiving an image to be detected, the image processor identifying an eye region from the image to be detected according to a plurality of facial feature points, the image processor performing image registration on the eye region to generate a normalized eye image to be detected, the deep learning processor extracting a plurality of eye features from the normalized eye image to be detected according to a deep learning model, and the deep learning processor outputting an eye state in the eye region according to the plurality of eye features and a plurality of training samples in the deep learning model.

In another embodiment of the invention, an eye state detection system comprising an image processor and a deep learning processor is provided.

The image processor is used to receive an image to be detected, identify an eye region from the image to be detected according to a plurality of facial feature points, and perform image registration on the eye region to generate a normalized eye image to be detected.

The deep learning processor is used to extract a plurality of eye features from the normalized eye image to be detected according to a deep learning model, and output an eye state in the eye region according to the plurality of eye features and a plurality of training samples in the deep learning model.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic diagram of a method of operating an eye state detection system according to one embodiment of the invention.

FIG. 2 shows an image to be detected.

FIG. 3 shows an eye image to be detected and generated by the image processor in FIG. 1 according to an eye region.

FIG. 4 is a flowchart of a method of operating the eye state detection system in FIG. 1.

### DETAILED DESCRIPTION

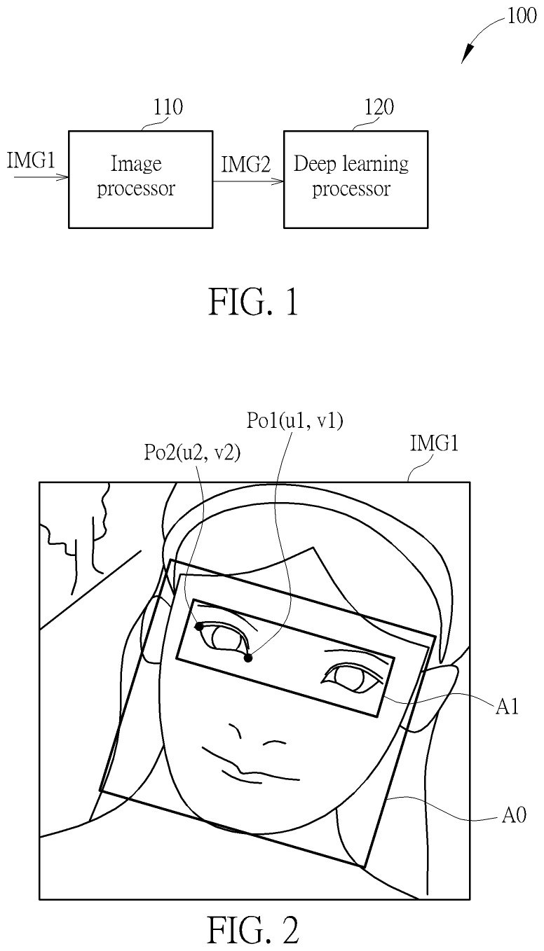

FIG. 1 is a schematic diagram of a method of operating an eye state detection system 100 according to one embodiment of the invention. The eye state detection system 100 comprises an image processor 110 and a deep learning processor 120. The deep learning processor 120 can be coupled to the image processor 110.

The image processor 110 can receive an image to be detected IMG1. FIG. 2 shows an image to be detected IMG1. The image to be detected IMG1 can be an image photographed by a user, an image captured by an in-vehicle monitoring camera, and can be generated by other devices on the basis of various application fields. Further, in some embodiments of the invention, the image processor 110 can be an application-specific integrated circuit specific for image processing, or a general application processor for executing a corresponding procedure.

The image processor 110 can identify an eye region A1 from the image to be detected IMG1 according to a plurality of facial feature points. In some embodiments of the invention, the image processor 110 can first identify a facial region A0 from the image to be detected IMG1 according to the plurality of facial feature points, and then identify the eye region A1 from the facial region A0 according to a plurality of eye keypoints. The facial feature points can be parameter values associated with facial features default in the system. The image processor 110 can extract a parameter value for comparison from the image to be detected IMG1 by using the image processing technology, and compare the parameter values for comparison with facial features default in the system to identify whether a human face is present in the image to be detected IMG1. After the facial region A0 is detected, the image processor 110 can then detect the eye region A1 in the facial region A0. In this manner, when no human face is present in the image, the embodiment can prevent the image processor 110 from directly performing complicated computations as required for human eye detection.

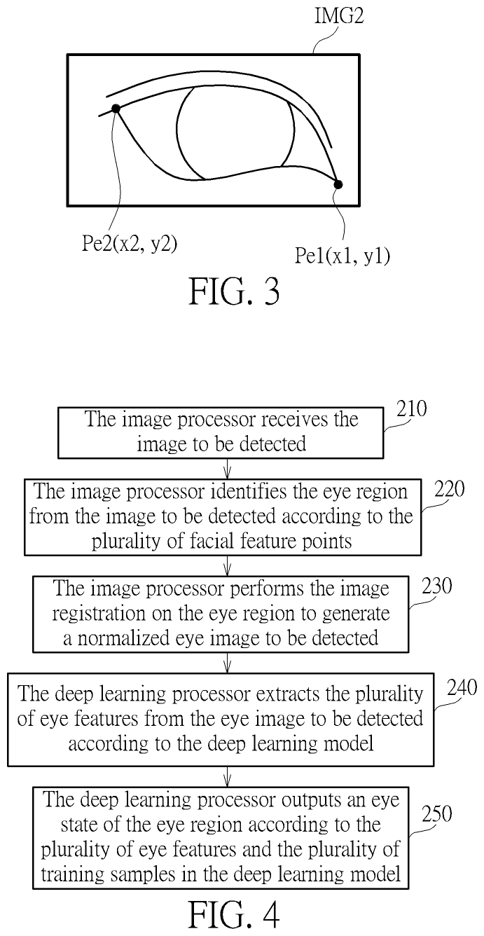

Indifferent or identical images to be detected, since the image processor 110 may identify different sizes of eye regions, the image processor 110 can perform image registration on the eye region A1 to generate normalized eye images to be detected, in order to facilitate a subsequent analysis performed by the deep learning processor 120, and prevent a false determination resulting from differences in eye sizes and angles in the images to be detected. FIG. 3 shows an eye image to be detected IMG2 and generated by the image processor 110 according to an eye region A1. For convenience of reference, in the embodiment of FIG. 3, the eye image to be detected IMG2 only includes a right eye in the eye region A1, and a left eye in the eye region A1 can be represented by another eye image to be detected. It should be clear that the invention is not limited to the configuration as shown in the embodiment. In another embodiment of the invention, the eye image to be detected IMG2 can include, depending on the requirement of a deep learning processor 120, both the left and right eyes in the eye region A1.

In the image to be detected IMG1, eye-corner coordinates in the eye region A1 can be represented by coordinates Po1 (u1,v1) and Po2 (u2,v2). In the eye image to be detected IMG2 generated after image registration, transformed eye-corner coordinates Pe1 (x1,y1) and Pe2 (x2,y2) generated after image registration correspond to the eye-corner coordinates Po1 (u1,v1) and Po2 (u2,v2). In some embodiments of the invention, locations of the transformed eye-corner coordinates Pe1 (x1,y1) and Pe2 (x2,y2) can be fixed in the eye image to be detected IMG2. The image processor 110 can transform, by performing an affine operation such as a shift, rotation, or scaling, the eye-corner coordinates Po1 (u1,v1) and Po2 (u2,v2) in the image to be detected IMG1 into transformed eye-corner coordinates Pe1 (x1,y1) and Pe2 (x2,y2) in the eye image to be detected IMG2. In other words, different affine transformation operations may be applied to different images to be detected IMG1 to perform transformation, to enable the eye region in the image to be detected IMG1 to stay at a fixed default location in the eye image to be detected IMG2, thereby achieving normalization by representing using a standard size and direction.

Since the affine transformation is primarily a first-order linear transformation between coordinates, the affine transformation can be represented by, for example, Formula 1 and Formula 2.

$$\begin{matrix}[ \begin{matrix}u 1 \\ v 1\end{matrix} ] = [ \begin{matrix}a & - b & dx \\ b & a & dy\end{matrix} ] [ \begin{matrix}x 1 \\ y 1 \\ 1\end{matrix} ] & Formula 1 \\ [ \begin{matrix}u 2 \\ v 2\end{matrix} ] = [ \begin{matrix}a & - b & dx \\ b & a & dy\end{matrix} ] [ \begin{matrix}x 2 \\ y 2 \\ 1\end{matrix} ] & Formula 2\end{matrix}$$

Since the eye-corner coordinates Po1 (u1,v1) and Po2 (u2,v2) can be transformed using a same operation into the eye-corner coordinates Pe1 (x1,y1) and Pe2 (x2,y2), an eye-corner coordinate matrix A can be defined according to the eye-corner coordinates Po1 (u1,v1) and Po2 (u2,v2). The eye-corner coordinate matrix A can be represented by Formula 3.

$$A = [ \begin{matrix}u 1 \\ v 1 \\ u 2 \\ v 2\end{matrix} ] = [ \begin{matrix}x 1 & - y 1 & 1 & 0 \\ y 1 & x 1 & 0 & 1 \\ x 2 & - y 2 & 1 & 0 \\ y 2 & x 2 & 0 & 1\end{matrix} ] [ \begin{matrix}a \\ b \\ dx \\ dy\end{matrix} ] \qquad Formula 3$$

That is, the eye-corner coordinate matrix A can be regarded as a multiplication result of a target transformed matrix B and an affine transformation parameter matrix C generated according to the eye-corner coordinates Pe1 (x1,y1) and Pe2 (x2,y2). The target transformed matrix B comprises the eye-corner coordinates Pe1 (x1,y1) and Pe2 (x2,y2), and can be represented by, for example, Formula 4. The affine transformation parameter matrix C can be represented by, for example, Formula 5.

$$\begin{matrix}B = [ \begin{matrix}x 1 & - y 1 & 1 & 0 \\ y 1 & x 1 & 0 & 1 \\ x 2 & - y 2 & 1 & 0 \\ y 2 & x 2 & 0 & 1\end{matrix} ] & Formula 4 \\ C = [ \begin{matrix}a \\ b \\ dx \\ dy\end{matrix} ] & Formula 5\end{matrix}$$

In the situation as such, the image processor 110 can obtain the affine transformation parameter matrix C using Formula 6 to transform between the eye-corner coordinates Po1 (u1,v1) and Po2 (u2,v2) and the eye-corner coordinates Pe1 (x1,y1) and Pe2 (x2,y2).

$$C = [ \begin{matrix}a \\ b \\ dx \\ dy\end{matrix} ] = {( {B}^{T} B )}^{- 1} {B}^{T} A \qquad Formula 6$$

That is, the image processor 110 can multiply a transpose B^{T} of the target transformed matrix B by the target transformed matrix B to produce a first matrix (B^{T}B), and multiply an inverse (B^{T}B)⁻¹ of the first matrix (B^{T}B) by the transpose B^{T} of the target transformed matrix B and the eye-corner coordinate matrix A to generate the affine transformation parameter matrix C. Consequently, the image processor 110 can process the eye region A1 using the affine transformation parameter matrix C to generate the eye image to be detected IMG2. The target transformed matrix B comprises two coordinate matrices of the eye-corner coordinate matrix A of the eye image to be detected.

After completion of the image registration and obtaining the eye image to be detected IMG2, the deep learning processor 120 is configured to extract a plurality of eye features from the eye image to be detected IMG2 according to a deep learning model, and output an eye state of the eye region according to the plurality of eye features and a plurality of training samples in the deep learning model.

For example, the deep learning model in the deep learning processor 120 can be a Convolution Neural Network (CNN). The convolution neural network comprises primarily a convolution layer, a pooling layer, and a fully connected layer. In the convolution layer, the deep learning processor 120 can perform a convolution operation on the eye image to be detected IMG2 using a plurality of feature detectors, also referred to as convolutional kernels, so as to extract various feature data from the eye image to be detected IMG2. Next, the deep learning processor 120 can reduce a noise in the feature data by selecting a local maximum value, flatten, via the fully connected layer, the feature data in the pooling layer, and connect to the a neural network trained and produced by the preliminary training samples.

Since the convolution neural network can compare different features on the basis of the preliminary training samples, and output a final determination result according to an association between different features, a state of eye opening or closing can be determined more accurately for various scenarios, postures, and ambient light, and reliability of the determined eye state can be output to serve as a reference for users.

In some embodiments of the invention, the deep learning processor 120 can be an application-specific integrated circuit specific for processing deep learning, and can be a general application processor or a general purpose graphic processing unit (GPGPU) for executing corresponding procedures.

FIG. 4 is a flowchart of a method 200 of operating the eye state detection system 100. The method 200 comprises Steps S210 through S250:

S210: the image processor 110 receives the image to be detected IMG1;

S220: the image processor 110 identifies the eye region A1 from the image to be detected IMG1 according to the plurality of facial feature points;

S230: the image processor 110 performs the image registration on the eye region A1 to generate a normalized eye image to be detected IMG2;

S240: the deep learning processor 120 extracts the plurality of eye features from the eye image to be detected IMG2 according to the deep learning model; and

S250: the deep learning processor 120 outputs an eye state of the eye region A1 according to the plurality of eye features and the plurality of training samples in the deep learning model.

In Step S220, the image processor 110 can first identify the facial region A0 using the plurality of human facial feature points, and then identify the eye region A1 using the plurality of eye keypoints. In other words, the image processor 110 can determine the eye region A1 from the facial region A0 after the facial region A0 is identified. In this manner, when no human face is present in the image, the embodiment can prevent the image processor 110 from directly performing complicated computations as required for human eye detection.

In addition, in order to prevent a false determination resulting from differences in eye sizes and angles in the images to be detected, in Step S230 of the operation method 200, an image registration process is performed to generate the normalized eye images to be detected IMG2. For instance, the operation method 200 can be employed to obtain, according to Formulas 3 through 6, the affine transformation parameter matrix C for transformation between the eye-corner coordinates Po1 (u1,v1) and Po2 (u2,v2) in the image to be detected IMG1 and the eye-corner coordinates Pe1 (x1,y1) and Pe2 (x2,y2) in the eye image to be detected IMG2.

In some embodiments of the invention, the deep learning model utilized in Steps S240 and S250 can comprise a convolutional neural network. Since the convolutional neural network can compare various features according to the preliminary training sample, and output the final determination result according to the association between various features, the state of eye opening or closing can be determined more accurately for various scenarios, postures, and ambient light, and the reliability of the determined eye state can be output to serve as a reference for users.

The eye state detection system and the operation method thereof as provided in the embodiments of the invention can be employed to normalize the eye region in the image to be detected by image registration, and determine the state of eye opening or closing more accurately using the deep learning model. Consequently, the eye closure detection can be more efficiently applied to a photographing function in various fields such as a driving auxiliary system or digital camera.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A method of operating an eye state detection system, the eye state detection system comprising an image processor and a deep learning processor, the method comprising:
the image processor receiving an image to be detected;
the image processor identifying an eye region from the image to be detected according to a plurality of facial feature points;
the image processor performing image registration on the eye region to generate a normalized eye image to be detected;
the deep learning processor extracting a plurality of eye features from the normalized eye image to be detected according to a deep learning model; and
the deep learning processor outputting an eye state of the eye region according to the plurality of eye features and a plurality of training samples in the deep learning model.

2. The method of claim 1, wherein the image processor identifying the eye region from the image to be detected according to the plurality of facial feature points comprises:
identifying a facial region from the image to be detected according to the plurality of facial feature points; and
identifying the eye region from the facial region according to a plurality of eye keypoints.

3. The method of claim 1, wherein the deep learning model comprises a convolutional neural network.

4. The method of claim 1, wherein the image processor performing image registration on the eye region to generate the normalized eye image to be detected comprises:
defining an eye-corner coordinate matrix of the eye region;
defining a target transformed matrix according to the eye-corner coordinate matrix, the target transformed matrix comprising transformed eye-corner coordinates of the normalized eye image to be detected;
multiplying the target transformed matrix by a transpose thereof to generate a first matrix;
multiplying an inverse of the first matrix, the transpose of the target transformed matrix, and the eye-corner coordinate matrix to generate an affine transformation parameter matrix; and
processing the eye region by using the affine transformation parameter matrix to generate the eye image to be detected.

5. The method of claim 4, wherein a product of the target transformed matrix and the affine transformation parameter matrix is the eye-corner coordinate matrix.

6. An eye state detection system comprising:
an image processor configured to receive an image to be detected, identify an eye region from the image to be detected according to a plurality of facial feature points, and perform image registration on the eye region to generate a normalized eye image to be detected; and
a deep learning processor configured to extract a plurality of eye features from the normalized eye image to be detected according to a deep learning model, and output an eye state of the eye region according to the plurality of eye features and a plurality of training samples in the deep learning model.

7. The eye state detection system of claim 6, wherein the image processor is configured to identify a facial region from the image to be detected according to the plurality of facial feature points, and identify the eye region from the facial region according to a plurality of eye keypoints.

8. The eye state detection system of claim 6, wherein the deep learning model comprises a convolutional neural network.

9. The eye state detection system of claim 6, wherein the image processor is configured to define an eye-corner coordinate matrix of the eye region, define a target transformed matrix according to the eye-corner coordinate matrix, multiply the target transformed matrix by a transpose thereof to generate a first matrix, multiply an inverse of the first matrix, the transpose of the target transformed matrix, and the eye-corner coordinate matrix to generate an affine transformation parameter matrix, and process the eye region by using the affine transformation parameter matrix to generate the eye image to be detected, the target transformed matrix comprising transformed eye-corner coordinates of the normalized eye image to be detected.

10. The eye state detection system of claim 9, wherein a product of the target transformed matrix and the affine transformation parameter matrix is the eye-corner coordinate matrix.
